(19) United States
(12) Patent Application Publication
(10) Pub. No.: US 2025/0204940 A1
(43) Pub. Date: Jun. 26, 2025

(54) EMBOLIC PROTECTION DEVICES AND METHODS FOR EMBOLIC PROTECTION

(71) Applicant: Boston Scientific Scimed, Inc., Maple Grove, MN (US)

(72) Inventor: Joseph Czyscon, Elk River, MN (US)

(73) Assignee: Boston Scientific Scimed, Inc., Maple Grove, MN (US)

(21) Appl. No.: 18/989,240

(22) Filed: Dec. 20, 2024

Related U.S. Application Data
(60) Provisional application No. 63/613,356, filed on Dec. 21, 2023.

(51) Int. Cl. A61B 17/22; A61B 17/00

(52) U.S. Cl. CPC A61B 17/22; A61B 17/00234; A61B 2017/00238; A61B 2017/00292; A61B 2017/00778; A61B 2017/22079; A61B 2217/005

(57) ABSTRACT

Medical devices and methods for using medical devices are disclosed. An example method of removing thrombotic material from a treatment site of a patient includes introducing an embolic protection device into the patient, deploying the embolic protection device adjacent the treatment site, introducing a thrombectomy device into the patient, conducting a thrombectomy procedure at the treatment site, withdrawing the thrombectomy device from the patient and withdrawing the embolic protection device from the patient.

### CROSS-REFERENCE TO RELATED APPLICATIONS

This application claims the benefit of priority under 35 U.S.C. § 119 of U.S. Provisional Application No. 63/613,356, filed Dec. 21, 2023, the entire disclosure of which is hereby incorporated by reference.

### BACKGROUND

Removal of tissue such as blood clots from within a vasculature may improve patient conditions and quality of life. Clot removal may be beneficial or even necessary to improve patient outcomes. For example, in the peripheral vasculature, interventions and procedures can reduce the need for an amputation by 80 percent. The ultimate goal of any technique to treat these conditions is to remove the blockage and to restore patency, quickly, safely, and cost effectively.

Thrombectomy is a procedure for removing thrombus from the vasculature of a patient. Mechanical and fluid-based systems can be used to remove thrombus. With fluid-based systems, an infusion fluid may be infused to a treatment area of a vessel with a catheter to dislodge the thrombus. In some instances, an effluent (e.g., the infusion fluid and/or blood) including the dislodged thrombus may be extracted (e.g., aspirated) from the vessel through the catheter. In other examples, a separate aspiration device may be utilized in conjunction with a thrombectomy system to extract effluent from a treatment area of a vessel.

Further, while thrombus removal systems may remove most thrombotic material from within a blood vessel, in some situations they may face challenges when removing large amounts of thrombotic material. Accordingly, in some instances it may be desirable to utilize an embolic protection filter to capture thrombotic material released during a thrombectomy procedure. Thus, there is a need for devices, including embolic protection devices that can be utilized in conjunction with mechanical and fluid-based thrombus removal systems during a thrombectomy procedure. Described herein are embolic protection devices and methods of using them to capture thrombotic debris released during a thrombectomy procedure.

### SUMMARY OF THE DISCLOSURE

This disclosure provides design, material, manufacturing method, and use alternatives for medical devices. An example method of removing thrombotic material from a treatment site of a patient includes introducing an embolic protection device into the patient, deploying the embolic protection device adjacent the treatment site, introducing a thrombectomy device into the patient, conducting a thrombectomy procedure at the treatment site, withdrawing the thrombectomy device from the patient and withdrawing the embolic protection device from the patient.

Alternatively or additionally to any of the embodiments above, wherein the embolic protection device is positioned upstream of the thrombectomy device.

Alternatively or additionally to any of the embodiments above, wherein the embolic protection device is introduced through a first femoral vein of the patient.

Alternatively or additionally to any of the embodiments above, wherein the thrombectomy device is introduced through a second femoral vein of the patient.

Alternatively or additionally to any of the embodiments above, wherein the embolic protection device is introduced through an internal or external jugular vein of the patient and wherein the thrombectomy device is introduced through a femoral vein of the patient.

Alternatively or additionally to any of the embodiments above, further comprising introducing an access sheath into a femoral vein of the patient, and wherein the embolic protection device is introduced into the vein of the patient through the access sheath, and wherein the access sheath may be configured to aspirate thrombotic material from the patient.

Alternatively or additionally to any of the embodiments above, wherein aspirating thrombotic material from the patient includes aspirating thrombotic material from the treatment site, the embolic protection device or both the treatment site and the embolic protection device.

Alternatively or additionally to any of the embodiments above, further comprising introducing an aspiration device into a femoral vein of the patient after the thrombectomy device has been withdrawn from the patient.

Alternatively or additionally to any of the embodiments above, further comprising advancing the aspiration device to a position adjacent the embolic protection device prior to the withdrawing the embolic protection device from the patient, and wherein the aspiration device is configured to aspirate thrombotic material from the embolic protection device.

Alternatively or additionally to any of the embodiments above, wherein the aspiration device is introduced through a first femoral vein of the patient, and wherein the embolic protection device is introduced through a second femoral vein of the patient.

Alternatively or additionally to any of the embodiments above, wherein the embolic protection device is introduced through a subclavian artery of the patient.

Alternatively or additionally to any of the embodiments above, further comprising introducing an access sheath into the femoral vein of the patient, and wherein both the embolic protection device and the aspiration device are introduced into the patient through the access sheath.

Another example method of removing thrombotic material from a treatment site of a patient includes introducing an access sheath into a femoral vein of the patient, introducing an embolic protection device into the patient, deploying the embolic protection device adjacent the treatment site, introducing a thrombectomy device into the patient, conducting a thrombectomy procedure at the treatment site of the patient, withdrawing the thrombectomy device from the patient, conducting an aspiration procedure at the treatment site of the patient and the embolic protection device and withdrawing the embolic protection device from the patient.

Alternatively or additionally to any of the embodiments above, wherein conducting an aspiration procedure at the treatment site of the patient includes advancing the access sheath to the treatment site and aspirating thrombotic material through a lumen of the access sheath.

Alternatively or additionally to any of the embodiments above, wherein the embolic protection device is introduced through a first femoral vein of the patient and wherein the thrombectomy device is introduced through a second femoral vein of the patient.

Alternatively or additionally to any of the embodiments above, wherein the embolic protection device is introduced through an internal or external jugular vein of the patient and wherein the thrombectomy device is introduced through a femoral vein of the patient.

Alternatively or additionally to any of the embodiments above, further comprising introducing an aspiration catheter into the patient through the access sheath after the thrombectomy catheter has been withdrawn from the patient.

Alternatively or additionally to any of the embodiments above, wherein both the embolic protection device and the aspiration device are introduced into the patient through the access sheath.

Another example method of removing thrombotic material from a treatment site of a patient includes introducing an access sheath into a first femoral artery of the patient, introducing an embolic protection device into the patient through the access sheath, deploying the embolic protection device adjacent the treatment site, introducing a thrombectomy device into a second femoral artery of the patient, conducting a thrombectomy procedure at the treatment site of the patient, withdrawing the thrombectomy device from the patient, introducing an aspiration device into the patient through the access sheath, conducting an aspiration procedure at the treatment site of the patient and the embolic protection device, withdrawing the aspiration device from the patient and withdrawing the embolic protection device from the patient.

The above summary of some embodiments is not intended to describe each disclosed embodiment or every implementation of the present disclosure. The Figures and Detailed Description, which follow, more particularly exemplify these embodiments.

### BRIEF DESCRIPTION OF THE DRAWINGS

A better understanding of the features and advantages of the methods and apparatuses described herein will be obtained by reference to the following detailed description that sets forth illustrative examples, and the accompanying drawings of which:

FIG. 1 illustrates an example embolic protection device;

FIG. 2 illustrates a handle of the embolic protection device of FIG. 1;

FIG. 3 illustrates a basket of the embolic protection device of FIG. 1 in an unexpanded configuration;

FIG. 4 illustrates a basket of the embolic protection device of FIG. 1 in an expanded configuration;

FIG. 5 illustrates the embolic protection device of FIG. 1 and a secondary medical device positioned within a patient;

FIG. 6 illustrates the embolic protection device of FIG. 1 and a secondary medical device positioned within a patient;

FIG. 7 illustrates the embolic protection device of FIG. 1 and a secondary medical device positioned within a patient;

FIG. 8 illustrates the embolic protection device of FIG. 1 extending within an access catheter;

FIG. 9 illustrates the embolic protection device of FIG. 1 and a secondary medical device extending within an access catheter and positioned within a patient;

FIG. 10 illustrates the embolic protection device of FIG. 1 and a secondary medical device extending within an access catheter;

FIG. 11 illustrates the embolic protection device of FIG. 1 and a secondary medical device positioned within a patient;

FIG. 12 illustrates the embolic protection device of FIG. 1 and a secondary medical device positioned within a patient.

### DETAILED DESCRIPTION

The following description should be read with reference to the drawings, which are not necessarily to scale, wherein like reference numerals indicate like elements throughout the several views. The detailed description and drawings are intended to illustrate but not limit the claimed disclosure. Those skilled in the art will recognize that the various elements described and/or shown may be arranged in various combinations and configurations without departing from the scope of the disclosure. The detailed description and drawings illustrate example embodiments of the claimed disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently-such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

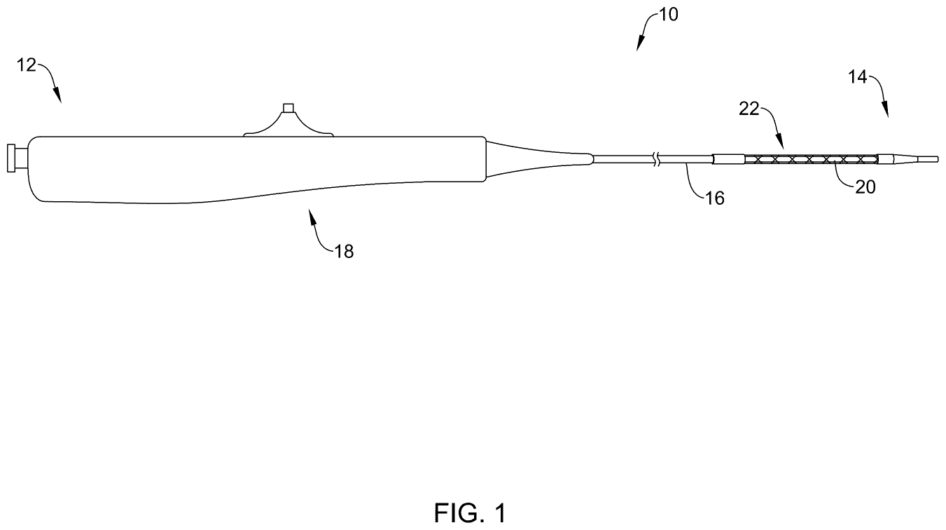

FIG. 1 illustrates an example embolic protection device 10. The embolic protection device 10 may include a proximal end region 12 and a distal end region 14. The proximal end region 12 of the embolic protection device 10 may include a handle 18. Further, the embolic protection device 10 may include an outer elongate shaft 16 that extends from a distal end of the handle 16 to the proximal end region 14 of the device. The embolic protection device 10 may further include an inner elongate member 20 that is slidably disposed within an outer elongate shaft 16. The outer elongate shaft 16 and the inner elongate member 20 may be flexible, to allow the embolic protection device 10 to navigate through tortious vessels within the body. Additionally, it can be appreciated that the embolic protection device 10 may include a lumen through which a guidewire or other elongate member may pass, such as a lumen through the inner elongate member 20.

In some examples, the overall length of the embolic protection device 10 may be about 30 cm (11.8 inches) to about 200 cm (78.7 inches), or about 50 cm (19.7 inches) to about 180 cm (70.9 inches), or about 100 cm (39.3 inches) to about 150 cm (59.1 inches). For example, the length of the device shown in FIG. 1 may be approximately 134 cm (52.7 inches) ±5 cm (1.9 inches) and has an effective length of about 115 cm (45.3 inches) ±5 cm (1.9 inches). The length of the outer elongate shaft 16 may be about 20 cm (7.9 inches) to about 180 cm (70.9 inches), or about 40 cm (15.7 inches) to about 180 cm (70.9 inches), or about 50 cm (19.7 inches) to about 150 cm (59.1 inches). In some cases, the outer elongate shaft 16 may have an inner diameter of about 0.5 mm (0.019 inches) +/−0.025 mm (0.00098 inches) to about 1.5 mm (0.059 inches) +/−0.025 mm (0.00098 inches), or about 1.0 mm (0.039 inches). The outer diameter of the outer elongate shaft 16 may be about 2.2 mm (0.087 inches) to about 3.0 mm (0.118 inches), or about 2.5 mm (0.098 inches) to about 2.85 mm (0.112 inches) +/−0.025 mm (0.00098 inches).

The outer elongate shaft 16 shaft may be formed of any appropriate material (a non-limiting list of which is disclosed herein), including a polymeric material such as styrenic block copolymers (e.g., Kraton®), functionalized thermoplastic olefins, thermoplastic elastomeric alloys, thermoplastic polyurethanes (e.g., Estane®, Pellethane®), polyamide-based thermoplastic elastomers (e.g., Pebax®), polyester-based thermoplastic elastomers (e.g. Hytrel®), ionomeric thermoplastic elastomers (e.g., Surlyn®), and any combinations thereof.

As will be discussed in greater detail herein, the embolic protection device 10 may further include an expandable braided basket 22 (shown in a collapsed configuration in FIG. 1) positioned along the distal end region 14 of the device 10.

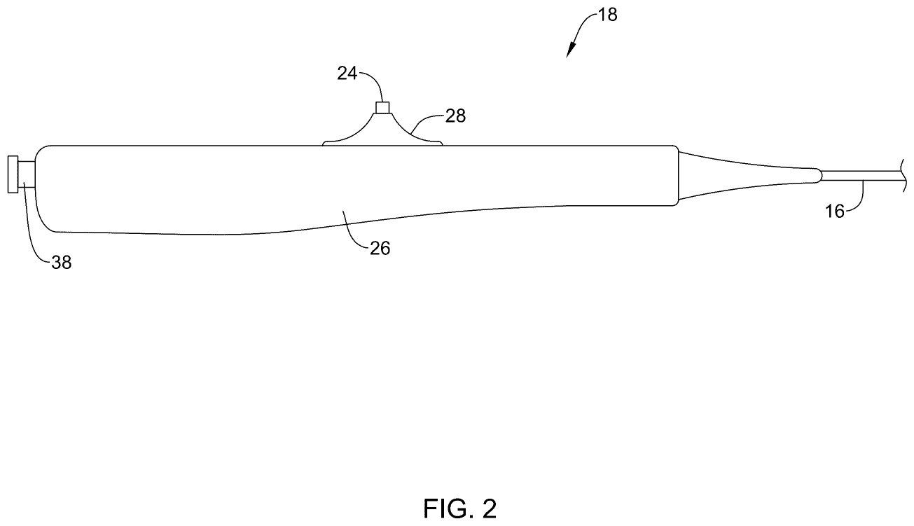

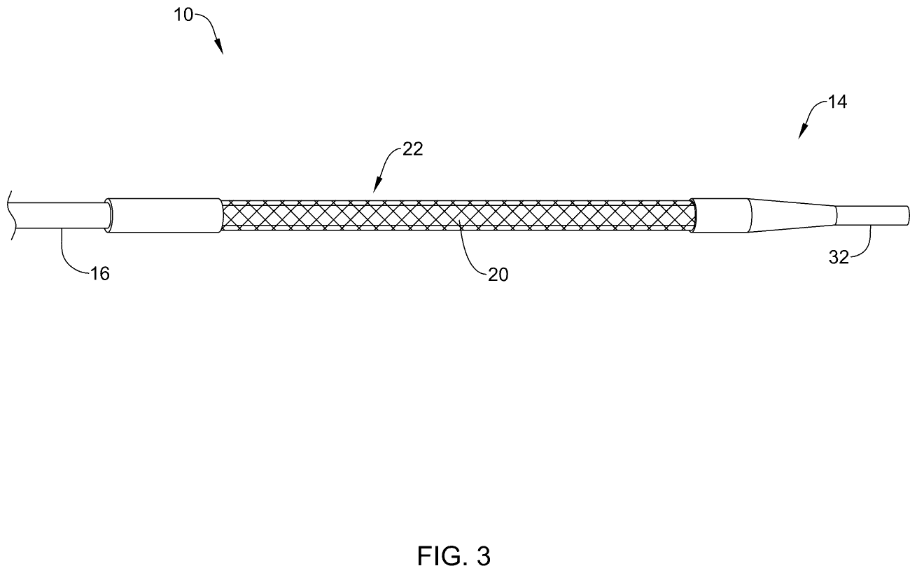

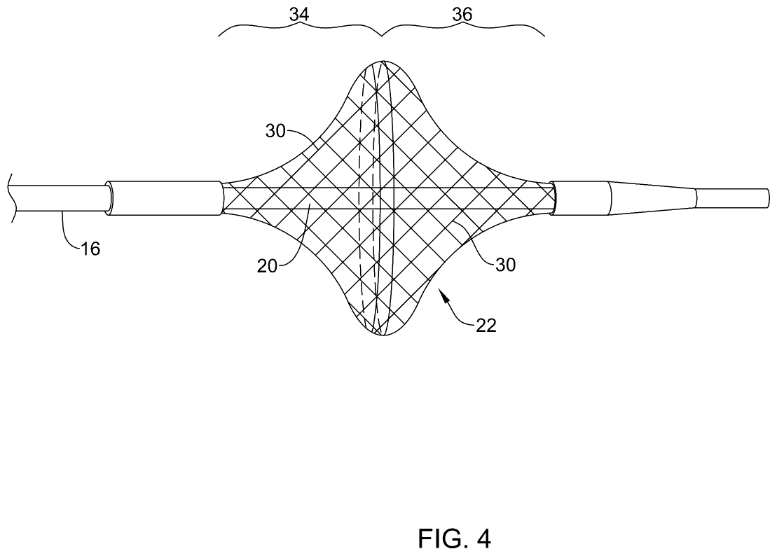

FIG. 2 illustrates the handle 18 of the embolic protection device 10 shown in FIG. 1. The distal end of the handle 18 may be coupled to the proximal end of the outer elongate member 16 and may include an actuating member 28 for operating the expandable basket 22 (e.g., expanding/contracting the expandable basket, and/or locking the expandable basket in a particular expansion position). As will be discussed in greater detail with respect to FIGS. 3-4, the actuation member 28 illustrated in FIG. 2 may include a slider that may extend or retract the inner elongate member 20 relative to the outer elongate shaft 16 to expand or contract the basket 22. The actuation member 28 may be configured to slide the inner elongate member 20 relative to the outer elongate shaft 16 to expand the expandable braided basket 22 from an unexpanded configuration to an expanded configuration. The handle 18 may also include a lock control 24, that, when depressed, allows the actuation member to move along a housing 26 of the handle 18 (e.g., the lock control 24 may include a button that releases a ratcheting lock to allow the actuation member 28 to move along the housing 26). Further, it can be appreciated that when the lock control 24 is released, it may lock the actuation member 28 (and thus the expansion of the basket 22) in place. The actuation member 28 can have ratcheted detents on the slider that provide tactical and auditory feedback to a user that the inner elongate member 20 is extending or retracting relative to the outer elongate shaft 16 to expand or retract the basket 22.

In some examples, the handle 18 may also include an ergonomic grip configured to be held by the user, e.g., doctor, nurse, technician, etc. Additionally, the embolic protection device 10 may further include a manifold 38 (e.g., port, luer port, etc.) positioned along a distal end region of the handle 18. The manifold 38 may be coupled to the inner elongate member 20. Thus, the manifold 38 may provide access into the lumen of the inner elongate member 20. It can be appreciated that the manifold 38 may be configured to permit a guidewire to pass into the lumen of the inner elongate member 20 and/or may be used for passing a fluid through the lumen of the inner elongate member 20 for flushing of the embolic protection device 10.

FIGS. 3-4 illustrate the braided basket 22 shifting between a first unexpanded configuration (shown in FIG. 3) and a second expanded configuration (shown in FIG. 4). FIG. 3 illustrates that a proximal end of the expandable braided basket 22 may be coupled to a distal end region of the outer elongate shaft 16 and a distal end of the expandable braided basket 22 may be coupled to the distal end region of the inner elongate member 20. As discussed herein, actuation (e.g., unlocking and sliding the actuation member 28 along the housing 26 shown in FIG. 2) of the actuation member 28 (shown in FIG. 2) may shift the basket 22 between the first unexpanded configuration (shown in FIG. 3) and a second expanded configuration (shown in FIG. 4). In some examples, the expandable braided basket 22 and/or the distal end region 14 of the embolic protection device 10 may also include one or more radiopaque makers 32. Inclusion of radiopaque markers 32 on the expandable braided basket 22 provide visual indication through fluoroscopy of unexpanded or expanded configurations.

FIG. 4 illustrates the embolic protection basket 22 in a fully deployed, expanded configuration. In this example, the braided wires may be wires (e.g., Nitinol, stainless steel, Inconel, etc.) that are configured to expand outwards to a maximum expansion diameter of about 10 mm (0.394 inches) to about 40 mm (1.57 inches), or about 15 mm (0.591 inches) to about 35 mm (1.38 inches), or about 20 mm (0.787 inches) to about 30 mm (1.81 inches). The basket 22 may have an unexpanded length of between 3 cm (1.81 inches) and 14 cm (5.51 inches).

In general, the expandable braided basket 22 may be formed of a plurality of braided filaments 30 (e.g., wires). In some instances, it may be beneficial to form the basket 22 from individual filaments 30 having a diameter of about 0.15 mm (0.006 inches) to about 0.20 mm (0.0079 inches) or more. This size of individual filament 30 may provide a basket 22 that, when braided, may capture thrombotic material flowing downstream of a thrombectomy and/or secondary medical treatment device. It can be appreciated that the basket 22 may also include one or more radiopaque markers disposed thereon. For examples, one or more of the individual filaments 30 may include a radiopaque marker disposed thereon.

As shown in FIG. 4, the basket 22 may be expanded by pulling the distal end of the basket 22 proximally (relative to the proximal end of the basket 22), such that the basket 22 expands radially outward. Alternatively, in some examples the basket 22 may be expanded outward by driving the proximal end distally. The basket 22 may be expanded partially or fully. In some examples, the basket 22 may be locked in one or more (or any/continuous) intermediate positions.

In some examples, the basket 22 may be formed of a plurality of wires of a

superelastic alloy (e.g., a nickel titanium alloy, such as Nitinol), which is superelastic and may be repeatedly bent without breaking. Other materials (stainless steel, polymers, etc.) may be used in some examples (a non-limiting list of which is disclosed herein). The basket 22 may be braided into a variety of different patterns. For example, the basket 22 may be braided into a pattern of diamond-shaped pores each having a pore area. The pore area may change as the basket 22 is expanded or contracted but may be consistent for a particular expansion diameter. In an embodiment, each diamond-shaped pore can range from greater than 1 millimeter (mm) (0.039 inches)×4 mm (0.157 inches) to less than 2 mm (0.079 inches)×5 mm (0.197 inches) in its width and length dimensions. Areas of the diamond-shaped pores would respectively range from 2 mm² (0.0031 inches²) to 5 mm² (0.0078 inches²) in these embodiments.

The proximal and distal ends of the basket 22 are bonded together with a polymer and attached to the inner elongate member 20 and the outer elongate shaft 16. In some examples, the polymer utilized to bond the proximal and distal ends of the basket 22 together may include polyimide or other suitable polymer. A non-limiting list of example polymers which may be utilized to bond the proximal and distal ends of the basket 22 together is disclosed herein.

FIG. 4 further illustrates that the basket 22 may be configured such that it includes a generally proximally facing portion 34 and a generally distally facing portion 36. In some examples, the proximally facing portion 34 and the distally facing portion 36 may include a pore area of 2 mm² (0.0031 inches²) to 5 mm² (0.0078 inches²). However, in other examples, the pore area of the proximally facing portion 34 may be different than the pore area of the distally facing portion 36. It can be appreciated that the area of the diamond-shaped pores of the proximal facing portion 34 may be configured to permit thrombus to enter the basket 22, while the area of the diamond-shaped pores of the distal facing portion 36 may be configured to trap thrombus, thereby preventing thrombotic material from passing distally beyond the basket 22. In other embodiments, it can be appreciated that the area of the diamond-shaped pores of the distal facing portion 36 may be configured to permit thrombus to enter the basket 22, while the area of the diamond-shaped pores of the proximal facing portion 34 may be configured to trap thrombus, thereby preventing thrombotic material from passing proximally beyond the basket 22.

In some examples, the basket 22 may be formed of a braided Nitinol material and may include 32 wires (32 ends) forming a pattern that is referred to as a half load diamond pattern (1 over 1 under 1). In other examples, the basket 22 may be formed of 48 end (48 wires) and is braided into a full load regular pattern (1 over 2 under 2).

In general, the basket 22 must be sufficiently stiff while in a vessel such that it can maintain its shape when expanded, such that it can capture thrombotic material downstream of a target treatment site. However, the basket 22 should not be overly stiff, or it may damage the vessel walls. The expandable basket 22 may therefore be configured so that it may be expanded against the vessel walls with a radial expansion force that is sufficient to engage the walls of the vessel, but yet be configured such that it does not damage the vessel walls. In some examples, the radial expansion force that is sufficient to expand the basket 22 against the walls of the vessel may be about 1 lbf (4.44 N) to about 4 lbf (17.79 N). Accordingly, it can be appreciated that the basket 22 may be configured such that the outer facing surfaces of the wires 30 are atraumatic. For example, the outer facing surfaces of the wires 30 may be smooth, such that the wires 30 do not anchor into the vessel wall. Rather, the basket 22 may be configured such that a clinician may track and position the basket 22 distal a target treatment site, easily expand the basket 22 such that it engages that vessel wall and easily collapse and remove the basket 22 (including any thrombotic material captured therein) from the vessel.

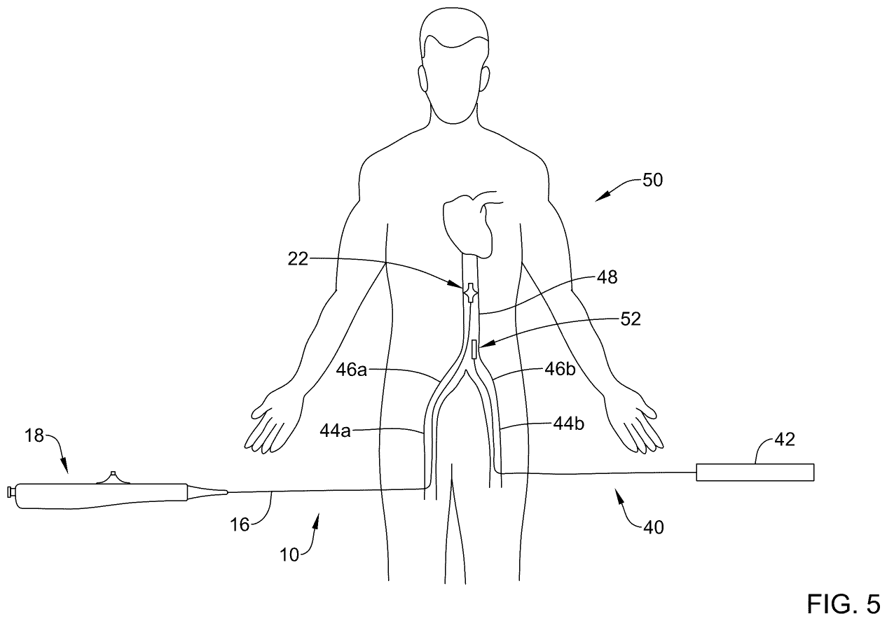

FIG. 5 illustrates the embolic protection device 10 and a secondary medical device (e.g., thrombectomy device, aspiration device, etc.) positioned within a patient 50. The embolic protection device 10 disclosed herein may be utilized for temporary placement within a patient 50. For example, FIG. 5 illustrates an example medical procedure in which the embolic protection device 10 is being utilized with a thrombectomy device 40.

FIG. 5 illustrates that the embolic protection device 10 may be first inserted into and tracked through the patient's right femoral vein 44a, through the patient's right iliac vein 46a, through the patient's IVC (inferior vena cava) 48, to a position proximate a target treatment site within the patient's IVC 48 (alternatively, the embolic protection device 10 may be inserted into and tracked through a patient's 50 left femoral vein 44b, through the patient's left iliac vein 46b and within the patient's IVC 48 and past a target treatment site and deployed distal to a target treatment site within the patient's IVC 48). In some examples, the embolic protection device 10 may be positioned upstream (relative to the direction of blood flow in the aorta 48) of the treatment site. In other examples, the embolic protection device 10 may be positioned downstream (relative to the direction of blood flow in the aorta 48) of the treatment site. As discussed herein, a clinician may utilize the handle 18 to shift the basket 22 from an unexpanded configuration to an expanded configuration.

After the embolic protection device 10 is positioned, the thrombectomy device 40 may be inserted into and tracked through a patient's 50 left femoral vein 44b, through the patient's left iliac vein 46b and within the patient's IVC 48, whereby the distal end region 52 of the thrombectomy device 40 may be positioned adjacent the target treatment site within the patient's left femoral vein 44b, left iliac vein 46b and/or the IVC 48 where the target thrombus is located (alternatively, the thrombectomy device 40 may be inserted into and tracked through a patient's 50 right femoral vein 44a, through the patient's right iliac vein 46a and within the patient's IVC 48, whereby the distal end region 52 of the thrombectomy device 40 may be positioned adjacent the target treatment site within the patient's right femoral vein 44a, right iliac vein 46a and/or the IVC 48 where the target thrombus is located 48). In some examples, the embolic protection device 10 may be positioned upstream (relative to the direction of blood flow in the IVC 48) of the treatment site. In other examples, the embolic protection device 10 may be positioned downstream (relative to the direction of blood flow in the IVC 48) of the treatment site.

The clinician may then utilize the handle 42 of the thrombectomy device 40 to operate the distal end region 52 of the thrombectomy device 40 to treat and/or remove thrombus from the target treatment site within the patient's left femoral vein 44b, left iliac vein 46b and/or the IVC 48 where the target thrombus is located 48. It can be appreciated that some thrombus may flow upstream and/or downstream during the thrombectomy procedure, wherein the thrombotic material may be trapped by the basket 22 of the embolic protection device 10. After the thrombectomy procedure is completed, the thrombectomy device 40 may be removed from the patient 50. Further, after the thrombectomy device 40 is removed, the clinician may utilize the handle 18 to collapse the basket 22 and remove the embolic protection device 10 (including the thrombus trapped in the basket 22) from the patient 50.

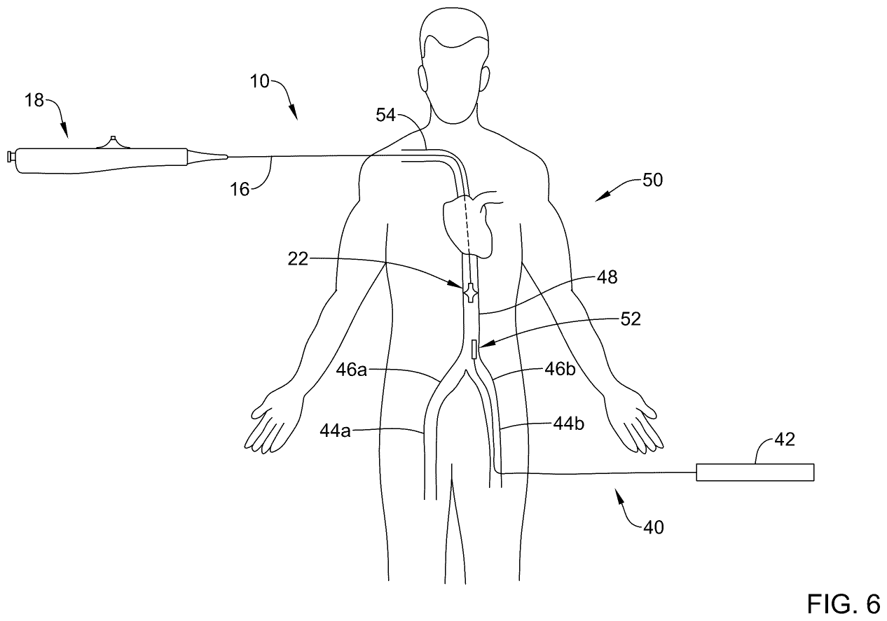

FIG. 6 illustrates the embolic protection device 10 and a secondary medical device (e.g., thrombectomy device, aspiration device, etc.) positioned within a patient 50. For example, FIG. 6 illustrates an example medical procedure in which the embolic protection device 10 is being utilized with a thrombectomy device 40. FIG. 6 illustrates that the embolic protection device 10 may be first inserted into the patient's internal or external jugular vein and tracked through the patient's brachiocephalic vein 54, into the superior vena cava to a position proximate a target treatment site within the patient's IVC 48. In some examples, the embolic protection device 10 may be positioned upstream (relative to the direction of blood flow in the IVC 48) of the treatment site. In other examples, the embolic protection device 10 may be positioned downstream (relative to the direction of blood flow in the IVC 48) of the treatment site. As discussed herein, a clinician may utilize the handle 18 to shift the basket 22 from an unexpanded configuration to an expanded configuration.

After the embolic protection device 10 is positioned, the thrombectomy device 40 may be inserted into and tracked through a patient's 50 left femoral vein 44b, through the patient's left iliac vein 46b and within the patient's IVC 48 (alternatively, the thrombectomy device 40 may be inserted into and tracked through a patient's 50 right femoral vein 44a, through the patient's right iliac vein 46a and within the patient's IVC 48), whereby the distal end region 52 of the thrombectomy device 40 may be positioned adjacent the target treatment site in the IVC 48. The clinician may then utilize the handle 42 of the thrombectomy device 40 to operate the distal end region 52 of the thrombectomy device 40 to treat and/or remove thrombus from the target treatment site with the left femoral vein 44b, left iliac vein 46b and IVC 48. It can be appreciated that some thrombus may flow upstream and/or downstream during the thrombectomy procedure, wherein the thrombotic material may be trapped by the basket 22 of the embolic protection device 10. After the thrombectomy procedure is completed, the thrombectomy device 40 may be removed from the patient 50. Further, after the thrombectomy device 40 is removed, the clinician may utilize the handle 18 to collapse the basket 22 and remove the embolic protection device 10 (including the thrombus trapped in the basket 22) from the patient 50.

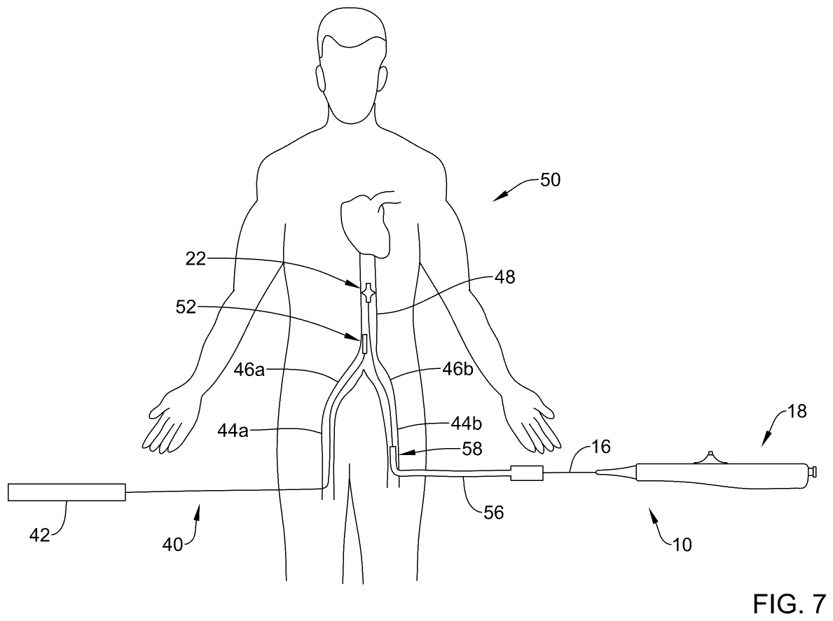

FIG. 7 illustrates the embolic protection device 10 and a secondary medical device (e.g., thrombectomy device, aspiration device, etc.) positioned within a patient 50. For example, FIG. 7 illustrates an example medical procedure in which the embolic protection device 10 is being utilized with a thrombectomy device 40.

FIG. 7 illustrates that an access sheath 56 may initially be inserted into a patient's 50 left femoral vein 44b, wherein by the distal end region 58 of the access sheath 56 points toward the patient's abdomen (alternatively, the access sheath 56 may initially be inserted into a patient's 50 right femoral vein 44a, wherein by the distal end region 58 of the access sheath 56 points toward the patient's abdomen).

FIG. 7 further illustrates that the embolic protection device 10 may be first inserted through the access sheath 56, tracked through the patient's left femoral vein 44b, through the patient's left iliac vein 46b, through the patient's IVC 48 and deployed proximate a target treatment site within the patient's IVC 48 (alternatively, the embolic protection device 10 may be first inserted through the access sheath 56, tracked through the patient's right femoral vein 44a, through the patient's right iliac vein 46a, through the patient's IVC 48 and past a target treatment site and deployed distal to a target treatment site within the patient's IVC 48). In some examples, the embolic protection device 10 may be positioned upstream (relative to the direction of blood flow in the IVC 48) of the treatment site. In other examples, the embolic protection device 10 may be positioned downstream (relative to the direction of blood flow in the IVC 48) of the treatment site. As discussed herein, a clinician may utilize the handle 18 to shift the basket 22 from an unexpanded configuration to an expanded configuration.

After the embolic protection device 10 is positioned, the thrombectomy device 40 may be inserted into and tracked through a patient's 50 right femoral vein 44a, through the patient's right iliac vein 46a and within the patient's IVC 48, whereby the distal end region 52 of the thrombectomy device 40 may be positioned adjacent the target treatment site within the patient's right femoral vein 44a, right iliac vein 46a, and IVC 48 (alternatively, the thrombectomy device 40 may be inserted into and tracked through a patient's 50 left femoral vein 44b, through the patient's left iliac vein 46b and within the patient's IVC 48, whereby the distal end region 52 of the thrombectomy device 40 may be positioned adjacent the target treatment site within the patient's left femoral vein 44b, left iliac vein 46b, and IVC 48). The clinician may then utilize the handle 42 of the thrombectomy device 40 to operate the distal end region 52 of the thrombectomy device 40 to treat and/or remove thrombus from the target treatment site within the patient's right femoral vein 44a, right iliac vein 46a, and IVC 48. It can be appreciated that some thrombus may flow upstream and/or downstream during the thrombectomy procedure, wherein the thrombotic material may be trapped by the basket 22 of the embolic protection device 10. After the thrombectomy procedure is completed, the thrombectomy device 40 may be removed from the patient 50. Further, after the thrombectomy device 40 is removed, the clinician may utilize the handle 18 to collapse the basket 22 and withdraw the embolic protection device 10 (including the thrombus trapped in the basket 22) through the access sheath 56 and remove it from the patient 50. After the embolic protection device 10 is removed from the patient 50, the access sheath 56 may be removed from the patient 50.

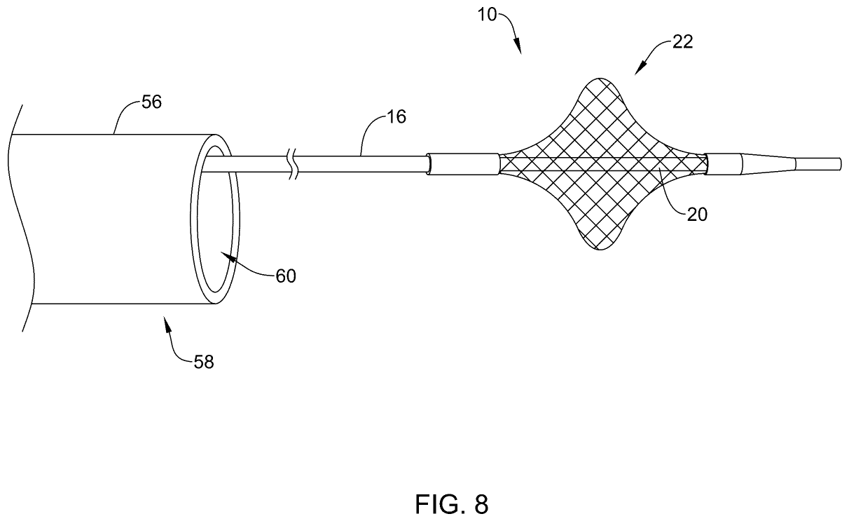

FIG. 8 illustrates the embolic protection device extending within a lumen 60 of the access sheath 56 (also shown in FIG. 7). In some examples, the access sheath 56 may be configured to aspirate thrombotic material through the lumen 60. For example, referring to the medical procedure described with respect to FIG. 7, after the thrombectomy device 40 is removed from the patient 50, the distal end 58 of the access sheath 56 may be advanced toward the basket 22 of the embolic protection device 10. Further, at any position during its advancement toward the embolic protection device 10, the access sheath 56 may aspirate thrombotic material through the lumen 60. Further yet, in some examples the access sheath 56 may be advanced to a position adjacent to the basket 22 of the embolic protection device 10, whereby the access sheath 56 may be utilized to aspirate thrombotic material out of the basket 22 of the embolic protection device 10.

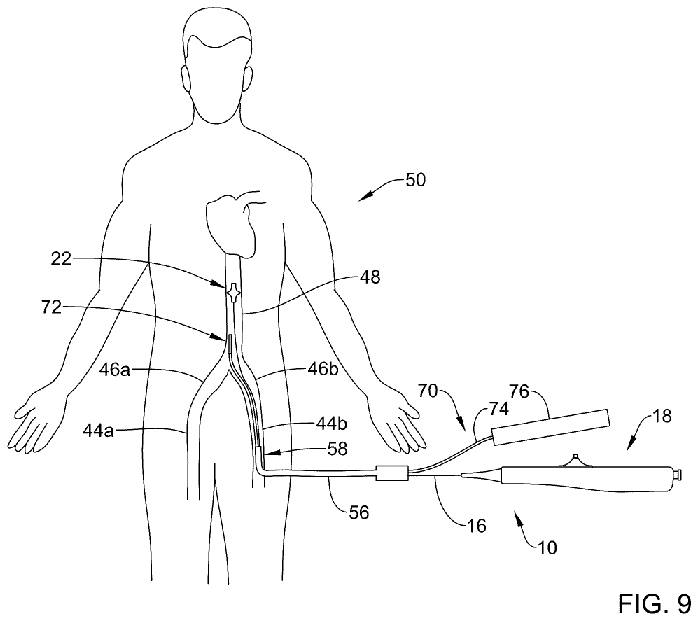

FIG. 9 illustrates the embolic protection device 10 and a secondary medical device (e.g., thrombectomy device, aspiration device, etc.) positioned within a patient 50. For example, FIG. 9 illustrates an example medical procedure in which the embolic protection device 10 is being utilized with both a thrombectomy device 40 (shown in FIGS. 5-7) and an aspiration device 70.

FIG. 9 illustrates that an access sheath 56 may initially be inserted into a patient's 50 left femoral vein 44b, wherein by the distal end region 58 of the access sheath 56 points toward the patient's abdomen. FIG. 9 further illustrates that the embolic protection device 10 may be first inserted through the access sheath 56, tracked through the patient's left femoral vein 44b, through the patient's left iliac vein 46b, through the patient's IVC 48 to a position proximate a target treatment site within the patient's IVC 48. As discussed herein, a clinician may utilize the handle 18 to shift the basket 22 from an unexpanded configuration to an expanded configuration.

After the embolic protection device 10 is positioned, a thrombectomy device 40 (shown in FIGS. 5-7) may be inserted into the patient (via the access sheath 56, the left femoral vein 44b as described herein, or the right femoral vein 44a as described herein) whereby a thrombectomy procedure may be performed on the target treatment site. After the thrombectomy procedure is completed, the thrombectomy device 40 may be removed.

After the thrombectomy device 40 is removed from the body, FIG. 9 illustrates that the shaft 74 of an aspiration device 70 may be inserted through the access sheath 56, whereby the distal end 72 of the aspiration device 70 may be advanced toward the basket 22 of the embolic protection device 10. The same access sheath 56 may be used to separately insert the embolic protection device 10, the thrombectomy device 40 and/or the aspiration device 70. Thus, as shown in FIG. 9, the embolic protection device 10 and the aspiration device 70 may be inserted through the access sheath 56, whereby the handle 76 of the aspiration device 70 may be actuated to initiate aspiration while the embolic protection device 10 is positioned in the IVC 48. Although FIG. 9 illustrates the access sheath 56, the embolic protection device 10 and the aspiration device 70 inserted into the patient's left femoral vein 44b, it is contemplated that the access sheath 56, the embolic protection device 10 and the aspiration device 70 may be inserted into the patient's right femoral vein 44a.

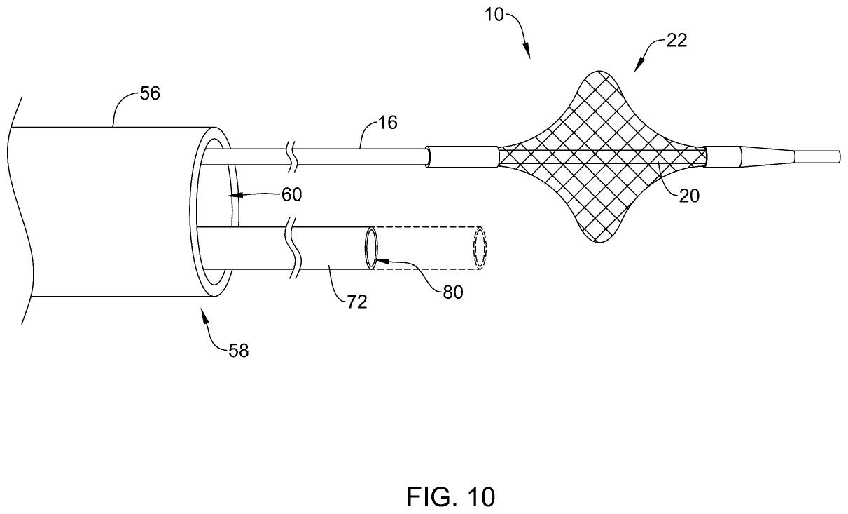

For example, FIG. 10 illustrates the aspiration device 70 positioned within and extending out of the distal end region 58 of the access sheath 56, whereby the distal end region 72 of the aspiration device 70 is being advanced through the patient's 50 vasculature toward the embolic protection device 10. At any position during its advancement toward the embolic protection device 10, the aspiration device 70 may aspirate thrombotic material through the lumen 80 of the shaft 74. For example, the aspiration device 70 may be used to aspirate thrombotic material from the target treatment site. Further yet, in some examples the aspiration device 70 may be advanced to a position adjacent to the basket 22 of the embolic protection device 10 (the advancement of the aspiration device 70 to a position adjacent to the basket 22 is depicted by the dashed lines in FIG. 10), whereby the aspiration device 70 may be utilized to aspirate thrombotic material out of the basket 22 of the embolic protection device 10. In yet other examples, the aspiration device 70 may be utilized to aspirate thrombotic material at the target treatment site while the thrombectomy procedure is being performed.

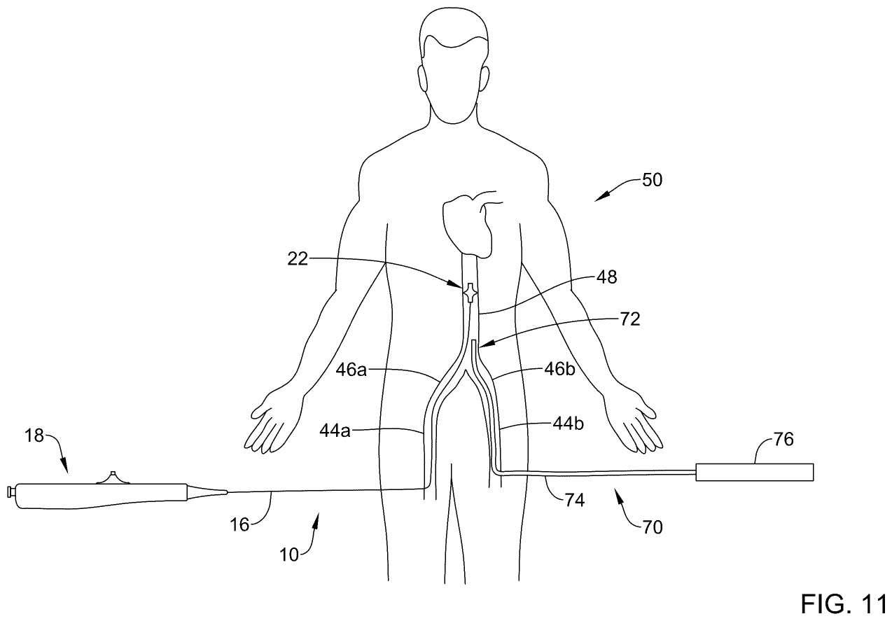

FIG. 11 illustrates the embolic protection device 10 and a secondary medical device (e.g., thrombectomy device, aspiration device, etc.) positioned within a patient 50. For example, FIG. 11 illustrates an example medical procedure in which the embolic protection device 10 may be utilized with both a thrombectomy device 40 (shown in FIGS. 5-7) and an aspiration device 70.

FIG. 11 illustrates that the embolic protection device 10 may be first inserted into and tracked through the patient's right femoral vein 44a, through the patient's right iliac vein 46a, through the patient's IVC 48 and deployed proximate a target treatment site within the patient's IVC 48 (alternatively, the embolic protection device 10 may be first inserted into and tracked through the patient's left femoral vein 44b, through the patient's left iliac vein 46b, through the patient's IVC 48 and past a target treatment site and deployed distal to a target treatment site within the patient's IVC 48). As discussed herein, a clinician may utilize the handle 18 to shift the basket 22 from an unexpanded configuration to an expanded configuration. After the embolic protection device 10 is positioned, a thrombectomy device 40 may be inserted into the patient (via the left femoral vein 44b as described herein) whereby a thrombectomy procedure may be performed at the target treatment site and the thrombectomy device 40 removed.

After the embolic protection device 10 is positioned, the shaft 74 an aspiration device 70 may be inserted through the patient's 50 left femoral vein 44b, whereby the distal end 72 of the aspiration device 70 may be advanced toward the basket 22 of the embolic protection device 10 (alternatively, the aspiration device 70 may be inserted through the patient's 50 right femoral vein 44a, whereby the distal end 72 of the aspiration device 70 may be advanced toward the basket 22 of the embolic protection device 10). For example, FIG. 11 illustrates the distal end region 72 of the aspiration device 70 being advanced through the patient's 50 vasculature toward the embolic protection device 10. At any position during its advancement toward the embolic protection device 10, the aspiration device 70 may aspirate thrombotic material through an inner lumen 80 (shown in FIG. 10) and out of the patient's 50 body. For example, the aspiration device 70 may be used to aspirate thrombotic material from the target treatment site. Further yet, in some examples the aspiration device 70 may be advanced to a position adjacent to the basket 22 of the embolic protection device 10, whereby the handle 76 of the aspiration device 70 may be actuated to initiate aspiration while the embolic protection device 10 is positioned in the IVC 48.

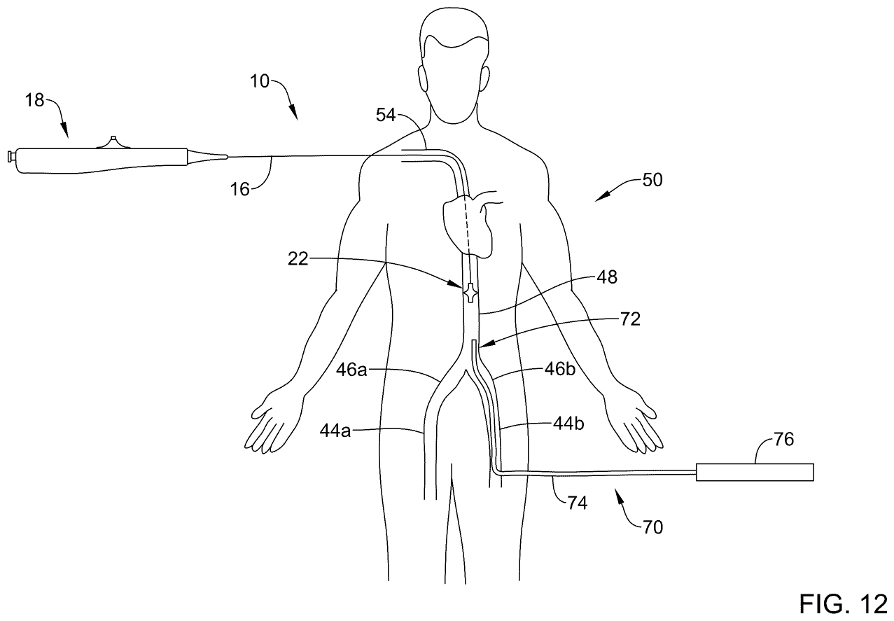

FIG. 12 illustrates the embolic protection device 10 and a secondary medical device (e.g., thrombectomy device, aspiration device, etc.) positioned within a patient 50. For example, FIG. 12 illustrates an example medical procedure in which the embolic protection device 10 may be utilized with both a thrombectomy device 40 (shown in FIGS. 5-7) and an aspiration device 70.

FIG. 12 illustrates that the embolic protection device 10 may be first inserted into and tracked through the patient's internal or external jugular vein and tracked through the patient's brachiocephalic vein 54, into the superior vena cava to a position proximate a target treatment site within the patient's IVC 48. As discussed herein, a clinician may utilize the handle 18 to shift the basket 22 from an unexpanded configuration to an expanded configuration. After the embolic protection device 10 is positioned, a thrombectomy device 40 may be inserted into the patient (via the left femoral vein 44b as described herein) whereby a thrombectomy procedure may be performed and the thrombectomy device 40 removed.

After the embolic protection device 10 is positioned, the shaft 74 an aspiration device 70 may be inserted through the patient's 50 left femoral artery 44b, whereby the distal end 72 of the aspiration device 70 may be advanced toward the basket 22 of the embolic protection device 10 (alternatively, the aspiration device 70 may be inserted through the patient's 50 right femoral artery 44a, whereby the distal end 72 of the aspiration device 70 may be advanced toward the basket 22 of the embolic protection device 10). For example, FIG. 12 illustrates the distal end region 72 of the aspiration device 70 is being advanced through the patient's 50 vasculature toward the embolic protection device 10. At any position during its advancement toward the embolic protection device 10, the aspiration device 70 may aspirate thrombotic material through an inner lumen 80 (shown in FIG. 10) and out of the patient's 50 body. For example, the aspiration device 70 may be used to aspirate thrombotic material from the target treatment site. Further yet, in some examples the aspiration device 70 may be advanced to a position adjacent to the basket 22 of the embolic protection device 10, whereby the handle 76 of the aspiration device 70 may be actuated to initiate aspiration while the embolic protection device 10 is positioned in the IVC 48.

The materials that can be used for the various components of the embolic protection system 10 and/or other devices disclosed herein may include those commonly associated with medical devices. For simplicity purposes, the following discussion refers to the embolic protection system 10 and its related components. However, this is not intended to limit the devices and methods described herein, as the discussion may be applied to other similar devices, tubular members and/or components of tubular members or devices disclosed herein.

The various components of the embolic protection system 10 and/or other devices disclosed herein may include a metal, metal alloy, polymer (some examples of which are disclosed herein), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

The various components of the embolic protection system 10 and/or other devices disclosed herein may include a polymer. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In at least some embodiments, portions or all of the embolic protection system 10 and/or other devices disclosed herein may be doped with, made of, or otherwise include a radiopaque material. Radiopaque materials are understood to be materials capable of producing a relatively bright image on a fluoroscopy screen or another imaging technique during a medical procedure. This relatively bright image aids the user of the pump/catheter assembly and its related components in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the embolic protection system 10 and its related components to achieve the same result.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A method of removing thrombotic material from a treatment site of a patient, the method comprising:
introducing an embolic protection device into the patient;
deploying the embolic protection device adjacent the treatment site;
introducing a thrombectomy device into the patient;
conducting a thrombectomy procedure at the treatment site;
withdrawing the thrombectomy device from the patient; and
withdrawing the embolic protection device from the patient.

2. The method of claim 1, wherein the embolic protection device is positioned upstream of the thrombectomy device.

3. The method of claim 2, wherein the embolic protection device is introduced through a first femoral vein of the patient.

4. The method of claim 3, wherein the thrombectomy device is introduced through a second femoral vein of the patient.

5. The method of claim 1, wherein the embolic protection device is introduced through an internal or external jugular vein of the patient and wherein the thrombectomy device is introduced through a femoral vein of the patient.

6. The method of claim 1, further comprising introducing an access sheath into a femoral vein of the patient, and wherein the embolic protection device is introduced into the vein of the patient through the access sheath, and wherein the access sheath may be configured to aspirate thrombotic material from the patient.

7. The method of claim 6, wherein aspirating thrombotic material from the patient includes aspirating thrombotic material from the treatment site, the embolic protection device or both the treatment site and the embolic protection device.

8. The method of claim 1, further comprising introducing an aspiration device into a femoral vein of the patient after the thrombectomy device has been withdrawn from the patient.

9. The method of claim 8, further comprising advancing the aspiration device to a position adjacent the embolic protection device prior to the withdrawing the embolic protection device from the patient, and wherein the aspiration device is configured to aspirate thrombotic material from the embolic protection device.

10. The method of claim 9, wherein the aspiration device is introduced through a first femoral vein of the patient, and wherein the embolic protection device is introduced through a second femoral vein of the patient.

11. The method of claim 8, wherein the embolic protection device is introduced through a subclavian artery of the patient.

12. The method of claim 8, further comprising introducing an access sheath into the femoral vein of the patient, and wherein both the embolic protection device and the aspiration device are introduced into the patient through the access sheath.

13. A method of removing thrombotic material from a treatment site of a patient, the method comprising:
introducing an access sheath into a femoral vein of the patient;
introducing an embolic protection device into the patient;
deploying the embolic protection device adjacent the treatment site;
introducing a thrombectomy device into the patient;
conducting a thrombectomy procedure at the treatment site of the patient;
withdrawing the thrombectomy device from the patient;
conducting an aspiration procedure at the treatment site of the patient and the embolic protection device; and
withdrawing the embolic protection device from the patient.

14. The method of claim 13, wherein conducting an aspiration procedure at the treatment site of the patient includes advancing the access sheath to the treatment site and aspirating thrombotic material through a lumen of the access sheath.

15. The method of claim 13, wherein the embolic protection device is introduced through a first femoral vein of the patient.

16. The method of claim 15, wherein the thrombectomy device is introduced through a second femoral vein of the patient.

17. The method of claim 13, wherein the embolic protection device is introduced through an internal or external jugular vein of the patient and wherein the thrombectomy device is introduced through a femoral vein of the patient.

18. The method of claim 13, further comprising introducing an aspiration catheter into the patient through the access sheath after the thrombectomy catheter has been withdrawn from the patient.

19. The method of claim 18, wherein both the embolic protection device and the aspiration device are introduced into the patient through the access sheath.

20. A method of removing thrombotic material from a treatment site of a patient, the method comprising:
introducing an access sheath into a first femoral artery of the patient;
introducing an embolic protection device into the patient through the access sheath;
deploying the embolic protection device adjacent the treatment site;
introducing a thrombectomy device into a second femoral artery of the patient;
conducting a thrombectomy procedure at the treatment site of the patient;
withdrawing the thrombectomy device from the patient;
introducing an aspiration device into the patient through the access sheath;
conducting an aspiration procedure at the treatment site of the patient and the embolic protection device;
withdrawing the aspiration device from the patient; and
withdrawing the embolic protection device from the patient.
